(19) United States
(12) Patent Application Publication
(10) Pub. No.: US 2019/0041374 A1
(43) Pub. Date: Feb. 7, 2019

(54) RAPID SCREENING METHOD OF PROCESSING RAW RICE FOR RICE PRODUCTS

(71) Applicant: Central South University of Forestry and Technology, Changsha (CN)

(72) Inventors: Qinlu LIN, Changsha (CN); Simin ZHAO, Changsha (CN); Yunhui CHENG, Changsha (CN); Yuqin DING, Changsha (CN); Lizhong LIN, Changsha (CN); Tao YANG, Changsha (CN); Huaxi XIAO, Changsha (CN); Wei WU, Changsha (CN); Yue WU, Changsha (CN)

(21) Appl. No.: 16/052,838

(22) Filed: Aug. 2, 2018

(30) Foreign Application Priority Data
Aug. 3, 2017 (CN) 201710654708.2

(51) Int. Cl. G01N 33/02; G06Q 10/06; G06Q 50/02; G06F 17/16; G06F 17/50

(52) U.S. Cl. CPC G01N 33/02; G06Q 10/06393; G06F 2217/16; G06F 17/16; G06F 17/5009; G06Q 50/02

(57) ABSTRACT

A rapid screening method of processing raw materials for rice products is disclosed. The invention establishes a membership function between the raw materials and the processing suitability of raw materials by adopting theories in fuzzy mathematics. In combination with the analytic hierarchy process to obtain the weight of each evaluation index, the invention then establishes a two-level evaluation model for evaluating the quality of the rice products to improve the scientificity and accuracy of rice products' quality evaluation. On the basis of the above, a mathematical model between the characteristics of raw materials and the comprehensive evaluation values of the quality of rice products is constructed through regression analyses, which can quantitatively calculate the suitability of different varieties of raw materials in the processing of rice products and can provide support for reasonable use of the raw materials.

### CROSS-REFERENCE TO RELATED APPLICATION

This application claims the benefit of priority under 35 U.S.C. § 119 to Chinese Patent Application No. CN201710654708.2, filed Aug. 3, 2017. The entire content of this application is hereby incorporated by reference herein.

### FIELD OF THE INVENTION

The present invention relates to the technical field of food processing, and particularly, to a rapid screening method of processing raw materials for rice products.

### DESCRIPTION OF THE PRIOR ART

Rice products, mainly made by rice and brown rice materials, primarily include rice noodles, glue puddings, rice dumplings, rice cakes, instant rice, rice puffed foods, glutinous fermented foods, and their derived products (such as fructose syrup, resistant starch, monosodium glutamate etc.). Suitable variety of rice materials is fundamental to producing high quality rice products. China has plenty variety of rice, tens of thousands of rice varieties as resources; significant differences in rice quality exist among different rice varieties, and the quality of rice products are closely related to their compositions and physicochemical characteristics. The processing requirements of raw materials to produce different rice products are different. How to rapidly select suitable raw materials from thousands of rice varieties for producing rice products is a problem needed to be solved immediately in the rice product processing industry.

The current methods of evaluating the suitability of raw materials for rice products are generally based on the correlation analysis, principal component analysis, regression analysis, etc., to establish a correlation between the physicochemical indexes of rice and the organoleptic quality of rice products, and then classify the raw materials by cluster analysis. The characteristics of each variety of raw materials in results of the cluster analysis further lead to evaluation criteria for processing suitability of rice. There are some deficiencies in current evaluation methods. First, there are too many physicochemical indexes of raw materials, among them there are certain correlations. The key indexes in these physicochemical ones of rice to affect the quality of rice products stay unclear. Second, the evaluation system of rice products was not well established. The quality of rice products is mainly determined by the total scores of sensory evaluations. Third, the current methods can only judge the suitability of raw materials for processing a certain type of rice products.

### SUMMARY OF THE INVENTION

The technical problem to be solved by the present invention is to provide a universal strategy for rapidly screening out raw materials for processing rice products, aiming at more accurately and rapidly screening raw materials for processing rice products.

A rapid screening method of processing raw materials for rice products comprises following steps: (1) collecting representative raw materials samples; (2) measuring physicochemical indexes of different varieties of raw materials; (3) producing rice products with different varieties of raw materials; (4) according to characteristics of each type of rice products, establishing an index set of multi-level assessment of the quality of rice products, obtaining weights of different indexes by utilizing analytic hierarchy process (AHP); (5) determining a membership of each evaluating index, constructing a fuzzy evaluation matrix; (6) obtaining fuzzy comprehensive evaluation values by conducting fuzzy matrix composite operations; (7) obtaining a mathematical model between the comprehensive evaluation values of rice products and the characteristics of raw materials by conducting regression analyses; (8) predicting processing suitability of different varieties of raw materials to produce rice products by adopting the mathematical model in the step (7).

Preferably, in the step (2), measure the physicochemical indexes of different varieties of raw materials, including: \textcircled{1} moisture, contents of protein and amylose of raw materials measured by a grain near infrared analyzer; \textcircled{2} taste values of raw materials measured by a taste meter; \textcircled{3} gelatinization parameters, including gelatinization temperature, peak viscosity, disintegration value, minimum viscosity, retrogradation value, final viscosity, and so forth, of raw materials through gelatinization tests after grinding and sifting.

Preferably, the step (4), according to the characteristics of each type of rice products, comprises: \textcircled{1} establishing an index set of multi-level assessment of the quality of rice products, including the rice products including rice noodles, instant rice, glue puddings, and rice sponge cakes; \textcircled{2} classifying the indexes for evaluating rice products quality into multi-level evaluation indexes; \textcircled{3} dividing first-level evaluation indexes into organoleptic quality index U₁, texture index U₂, and other physicochemical index U₃; \textcircled{4} obtaining second-level assessment indexes Uᵢⱼ below the first-level evaluation indexes Uᵢ, according to the characteristics of the rice noodles, the second-level evaluation indexes of the organoleptic quality index, including luster U₁₁, aroma U₁₂, morphology U₁₃, and mouthfeel U₁₄, namely, U₁={U₁₁, U₁₂, U₁₃, U₁₄}; the second-level evaluation indexes of the texture index, including elasticity U₂₁, viscidity U₂₂, hardness U₂₃, and chewiness U₂₄, namely, U₂={U₂₁, U₂₂, U₂₃, U₂₄}; the second-level evaluation indexes of the other physicochemical indexes, including pulping value U₃₁, broken rate U₃₂, namely, U₃={U₃₁, U₃₂}; \textcircled{5} obtaining the second-level evaluation indexes Uᵢⱼ under the first-level evaluation indexes Uᵢ, according to the characteristics of the instant rice; the second-level evaluation indexes of the organoleptic quality index, including luster before reconstitution U₁₁, morphology before reconstitution U₁₂, appearance U₁₃, mouthfeel U₁₄, and aroma U₁₅ after reconstitution, namely, U₁={U₁₁, U₁₂, U₁₃, U₁₄, U₁₅}; the second-level evaluation indexes of the texture index, including hardness U₂₁, adhesiveness U₂₂, elasticity U₂₃, chewiness U₂₄, and cohesiveness U₂₅, namely, U₂={U₂₁, U₂₂, U₂₃, U₂₄, U₂₅}; \textcircled{6} obtaining the second-level evaluation indexes Uᵢⱼ under the first-level evaluation indexes Uᵢ, according to the characteristics of the glue puddings; the second-level evaluation indexes of the organoleptic quality index, including appearance U₁₁, mouthfeel U₁₂, and soup cloudiness U₁₃, namely, U₁={U₁₁, U₁₂, U₁₃}; the second-level evaluation indexes of the texture index, including hardness U₂₁, elasticity U₂₂, adhesiveness U₂₃, resilience U₂₄, and chewiness U₂₅, namely, U₂={U₂₁, U₂₂, U₂₃, U₂₄, U₂₅};

the second-level assessment indexes of the other physicochemical indexes, including frozen cracking rate U₃₁, dehydration rate U₃₂, soup lucidity rate U₃₃, namely, U₃={U₃₁, U₃₂, U₃₃}; \textcircled{7} obtaining the second-level assessment indexes Uᵢⱼ under the first-level assessment indexes Uᵢ, according to the characteristics of the rice sponge cakes; the second-level assessment indexes of the organoleptic quality index, including morphology U₁₁, luster U₁₂, and aroma U₁₃, taste U₁₄, and mouthfeel U₁₅, namely, U₁={U₁₁, U₁₂, U₁₃, U₁₄, U₁₅}; the second-level assessment indexes of the texture index, including hardness U₂₁, elasticity U₂₂ adhesiveness U₂₃, resilience U₂₄, and chewiness U₂₅, namely, U₂={U₂₁, U₂₂, U₂₃, U₂₄, U₂₅}; the second-level assessment indexes of the other physicochemical indexes, including specific volume U₃₁, titration acidity U₃₂, namely, U₃={U₃₁, U₃₂}.

Preferably, in the step (4), obtaining weights of different evaluation indexes by conducting analytical hierarchy process (AHP), including:

according to experts' scoring results, obtaining judgment matrixes of the second-level evaluation index and the first-level evaluation index, normalizing the judgment matrixes, calculating second-weight of the second-level evaluation indexes wᵢⱼ and first-weight of the first evaluation indexes wᵢ, obtaining weight sets w₁={w₁₁, w₁₂, w₁₃, . . . , w₁ⱼ}, w₂−{w₂₁, w₂₂, w₂₃, . . . , w₂ⱼ}, w₃={w₃₁, w₃₂, w₃₃, . . . , w₃ⱼ}, w={w₁, w₂, w₃, . . . , wᵢ}.

Preferably, in the step (5), determining memberships of each evaluation index, establishing fuzzy evaluation matrixes, including: \textcircled{1} dividing the evaluation indexes in the step (4) further into ascending quantitative evaluation indexes, appropriate interval quantitative indexes, and descending quantitative indexes; \textcircled{2} calculating memberships of each second-level indexes; wherein for the ascending quantitative indexes, the general membership function for the corresponding level is:

$$r = \{ \begin{matrix}0 & {x}_{ij} \leq min ( {x}_{ij} ) \\ \frac{{x}_{ij} - min ( {x}_{ij} )}{max ( {x}_{ij} ) - min ( {x}_{ij} )} & min ( {x}_{ij} ) < {x}_{ij} < max ( {x}_{ij} ) \\ 1 & {x}_{ij} \geq max ( {x}_{ij} )\end{matrix}$$

for the descending quantitative index, the general membership function for the corresponding level is:

$$r = \{ \begin{matrix}1 & {x}_{ij} \leq min ( {x}_{ij} ) \\ \frac{max ( {x}_{ij} ) - {x}_{ij}}{max ( {x}_{ij} ) - min ( {x}_{ij} )} & min ( {x}_{ij} ) < {x}_{ij} < max ( {x}_{ij} ) \\ 0 & {x}_{ij} \geq max ( {x}_{ij} )\end{matrix}$$

for the appropriate interval quantitative index, the general membership function for the corresponding level is:

$$r = \{ \begin{matrix}1 & {s}_{1} \leq {x}_{ij} \leq {s}_{2} \\ \frac{{x}_{ij} - min ( {x}_{ij} )}{{s}_{1} - min ( {x}_{ij} )} & min ( {x}_{ij} ) < {x}_{ij} < {s}_{1} \\ \frac{max ( {x}_{ij} ) - {x}_{ij}}{max ( {x}_{ij} ) - {s}_{2}} & {s}_{2} < {x}_{ij} < max ( {x}_{ij} ) \\ 0 & {x}_{ij} > max ( {x}_{ij} ) , {x}_{ij} < min ( {x}_{ij} ) ,\end{matrix}$$

wherein xᵢⱼ represents the measurements of the second-level indexes, min(xᵢⱼ) and max(xᵢⱼ) represent the minimum and maximum values, respectively, and s₁ and s₂ represent the lower limit of the best value and the upper limit of the best value, respectively; \textcircled{3} establishing a single factor fuzzy matrix R:

$$R = \{ {r}_{ij} \} = [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ]$$

Preferably, in the step (6), obtaining fuzzy comprehensive evaluation value by conducting fuzzy matrix composite operation, including a multiplication of the single factor fuzzy matrix and weights of indexes determined in the step (4).

$$Y = w ∘ R = [ \begin{matrix}{w}_{1} & {w}_{2} & {w}_{3}\end{matrix} ] ∘ [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ]$$

wherein ∘ represents operations, different fuzzy operators are adopted according to the situations and operation results.

Preferably, in the step (6), using operator M(⋅, ⊕) in all calculations of the fuzzy matrix composite operations (⋅ and ⊕ represent algebraic product and sum of the fuzzy set, respectively).

Preferably, in the step (7), obtaining the mathematical model Y=A·xᵢ^{b}^{ᵢ} between the comprehensive evaluation values of the rice products such as rice noodles, glue puddings, instant rice, and rice sponge cakes and the characteristics of raw materials, wherein Y represents comprehensive values of the rice products, A is type-related coefficient of the rice products, xᵢ represents the physicochemical indexes of raw materials, such as moisture, amylose, gelatinization temperature, and taste value; bᵢ represents exponents, i=1, 2, 3 . . . .

Preferably, in the step (8), predicting the processing suitability of raw materials by using the mathematical model in the step (7), including: measuring the physicochemical indexes of raw materials, including using the grain near infrared analyzer to measure contents of moisture, protein and amylose of raw materials; obtaining the taste values with the taste meter; obtaining the gelatinization parameters, such as gelatinization temperatures, peak viscosity, disintegration values, minimum adhesiveness, retrogradation values, and final adhesiveness; substituting the aforesaid physicochemical indexes of raw materials into the mathematical model Y=A·xᵢ^{b}^{ᵢ} as defined in step (7), obtaining the comprehensive evaluation values of corresponding rice products quality, and evaluating the processing suitability of raw materials.

The invention establishes a membership function between the raw materials and the processing suitability of raw materials for processing rice products by adopting theories in fuzzy mathematics. In combination with the analytic hierarchy process to obtain the weight of each evaluation index, the invention then establishes a two-level evaluation model for evaluating the quality of the rice products to improve the scientificity and accuracy of rice products' quality evaluation. On the basis of the above, a mathematical model between the characteristics of raw materials and the comprehensive evaluation values of the quality of rice products is constructed through regression analyses, which can quantitatively calculate the suitability of different varieties of raw materials in the processing of rice products and can provide support for reasonable use of the raw materials.

### DETAILED DESCRIPTION OF THE PREFERRED EMBODIMENTS

The present invention will be further described with reference to the following embodiments.

### Embodiment 1

A rapid screening method of processing raw materials for rice products, particularly including the following steps:

S1: To collect representative raw materials samples, relevant sample information is shown in Table 1.

TABLE 1
Serial Number and name of Rice Varieties

| No. | Sample Name |
|---|---|
| 01 | 00884 |
| 02 | 8414 Nuo |
| 03 | K You 6 |
| 04 | Q Nuo 2 |
| 05 | R4132 Nuo |
| 06 | T You 100 |
| 07 | T You 1128 |
| 08 | T You 115 |
| 09 | T You 207 |
| 10 | T You 272 |
| 11 | T You 597 |
| 12 | Y Liangyou 1 |
| 13 | Y Liangyou 7 |
| 14 | Baofeng 2 |
| 15 | Bo II You15 |
| 16 | Boyou Shuangqing |
| 17 | Changguidao |
| 18 | Changnuo 468 |
| 19 | Chengnuo 88 |
| 20 | Enuo 9 |
| 21 | Fujing 2103 |
| 22 | Ganxin 203 |
| 23 | Guichao |
| 24 | Guiyou 2 |
| 25 | Hongnuo 19 |
| 26 | Jijing 94 |
| 27 | Jinongda 45 |
| 28 | Jinyou 163 |
| 29 | Jinyou 967 |
| 30 | Jinyou 978 |
| 31 | Jindao 8 |
| 32 | Jingxian 89 |
| 33 | Liangyou 6326 |
| 34 | Liaoxing 1 |
| 35 | Maoyou 601 |
| 36 | Meichi 8 |
| 37 | Mengliangyou 838 |
| 38 | Qianyou107 |
| 39 | Qianyou 568 |
| 40 | Qiuguang |
| 41 | Rongyou 7 |
| 42 | Rongyou 9 |
| 43 | Shenyou 9723 |
| 44 | Shenyou 9734 |
| 45 | Shenxian 6 |
| 46 | Shugeng |
| 47 | Tianfengyou 316 |
| 48 | Tianyou 103 |
| 49 | Tianyou 122 |
| 50 | Tianyou 998 |
| 51 | Wanjinyou 122 |
| 52 | Weiyou 277 |
| 53 | Weiyou 46 |
| 54 | Weiyou 644 |
| 55 | Weiyou 647 |
| 56 | Weiyouwan 3 |
| 57 | Wufengyou 998 |
| 58 | Wufengyou T025 |
| 59 | Wuyou 308 |
| 60 | Xianhe Dali |
| 61 | Xianong 2 |
| 62 | Xiangnuo |
| 63 | Xiangzaoxian 06 |
| 64 | Xiangzaoxian 24 |
| 65 | Xiangzaoxian 32 |
| 66 | Xiangzaoxian 42 |
| 67 | Xiangzaoxian 45 |
| 68 | Xindai |
| 69 | Xinnian 3 |
| 70 | Yangdao 6 |
| 71 | You 1686 |
| 72 | Youyou 128 |
| 73 | Yueyou 72 |
| 74 | Yunhui 290 |
| 75 | Zhanyou 2009 |
| 76 | Zhanyou 226 |
| 77 | Zhanyou 809 |
| 78 | Changbai 9 Fushun |
| 79 | Changbai 9 Jilin |
| 80 | Zhefu 802 |
| 81 | Zhennuo |
| 82 | Zhennuo 4130 |
| 83 | Zhong 9 You 838 |
| 84 | Zhongjiazao 32 |
| 85 | Zhongyou 106 |
| 86 | Zhongyou 9918 |
| 87 | Zhuliangyou 176 |
| 88 | Zhuliangyou 819 |

S2: To measure the physicochemical indexes of different rice varieties; to measure the content of moisture, protein and amylose of raw rice with a grain near-infrared analyzer; to measure the taste value with a taste meter; to measure the gelatinization properties and obtain parameters of gelatinization properties such as gelatinization temperature (GTP), peak viscosity (PV), disintegration value (DV), minimum viscosity (MV), retrogradation value (RGV), and final viscosity (FV), as shown in Table 2.

TABLE 2
Statistics of the physicochemical characteristics
of 88 Rice Varieties

| Physicochemical Indexes | AV | SD | Max | Min | Range |
|---|---|---|---|---|---|
| Moisture/% | 12.00 | 1.66 | 16.00 | 8.20 | 7.80 |
| Amylose/% | 17.64 | 6.07 | 27.00 | 0.60 | 26.40 |
| Protein/% | 9.43 | 1.58 | 13.90 | 5.68 | 8.22 |
| Taste Value | 50.50 | 9.47 | 81.00 | 38.00 | 43.00 |
| PV/(Pa · s) | 2.65 | 1.02 | 4.65 | 0.12 | 4.53 |
| MV/(Pa · s) | 1.51 | 0.68 | 3.29 | 0.05 | 3.24 |
| AV/(Pa · s) | 1.15 | 0.50 | 2.53 | 0.05 | 2.48 |
| FV/(Pa · s) | 2.74 | 1.10 | 6.37 | 0.06 | 6.31 |
| RGB/(Pa · s) | 0.49 | 0.37 | 2.39 | 0.02 | 2.37 |
| Peak Time/min | 5.72 | 0.55 | 6.47 | 3.13 | 3.34 |
| GTP/° C. | 77.81 | 5.64 | 88.60 | 64.45 | 24.15 |

AV = average
SD = standard deviation

S3: To produce dehydrated instant rice by using different varieties of raw materials, to measure the texture parameters and the scores of organoleptic quality of the dehydrated instant rice. A basic statistical result of the quality of instant rice is shown in Table 3.

TABLE 3
The Statistics of Dehydrated Instant Rice
Produced from Different Rice Variety

| | Mean | SD | Max | Min | Range |
|---|---|---|---|---|---|
| Hardness | 3156.32 | 102.82 | 5151.30 | 1499.90 | 3651.40 |
| Adhesiveness | 4.63 | 0.67 | 22.10 | 17.13 | 39.23 |
| Elasticity | 2.78 | 0.36 | 12.72 | 2.95 | 15.67 |
| Chewiness | 2618.36 | 123.81 | 5989.70 | 1080.90 | 4908.80 |
| Colloidity | 2403.24 | 89.57 | 4200.60 | 1094.60 | 3106.00 |
| Cohesiveness | 0.75 | 0.01 | 0.98 | 0.55 | 0.43 |
| Resilience | 1.27 | 0.02 | 1.52 | 0.30 | 1.22 |
| Luster | 3.87 | 0.11 | 4.80 | 1.00 | 3.80 |
| Morphology | 5.94 | 0.24 | 9.00 | 1.00 | 8.00 |
| Appearance | 3.63 | 0.10 | 4.80 | 1.00 | 3.80 |
| Mouthfeel | 6.89 | 0.19 | 9.00 | 0.00 | 9.00 |
| Aroma | 3.44 | 0.08 | 4.30 | 2.00 | 2.30 |

S4: To establish an index set of multi-level assessment, according to the product features of dehydrated instant rice is shown in Table 4.

TABLE 4
Comprehensive Quality Evaluation Index
System of the Dehydrated Instant Rice

| Overall | | | Weight of |
|---|---|---|---|
| Evaluation | | Specific Evaluation | Evaluation Factor |
| Objective | Sub-object (Uᵢ) | Index (Uᵢⱼ) | (w) |
| Comprehensive | Organoleptic | Luster before | 0.0659 |
| Quality of | Quality (Uᵢ) | reconstitution (U₁₁) | |
| Dehydrated | | Morphology before | 0.0529 |
| Instant Rice | | reconstitution (U₁₂) | |
| (Y) | | Appearance after | 0.0823 |
| | | reconstitution (U₁₃) | |
| | | Appearance after | 0.4155 |
| | | reconstitution (U₁₄) | |
| | | Aroma after | 0.1965 |
| | | reconstitution (U₁₅) | |
| | Texture (U₂) | Hardness (U₂₁) | 0.0333 |
| | | Adhesiveness (U₂₂) | 0.0150 |
| | | Elasticity (U₂₃) | 0.0506 |
| | | Chewiness (U₂₄) | 0.0746 |
| | | Cohesiveness (U₂₅) | 0.0134 |

To determine the weight (w) of each index (Uᵢⱼ) by conducting the analysis hierarchy process, detailed steps are included as follows:

First, according to the experts' scoring results, obtain judgment matrixes of the first-level evaluation index and the second-level evaluation index, pairly compare the indexes in the same-level, make a relative significance judgment, conducting 1-9 scale method (Table 5), an estimated value of the relative significance of the i^{th} index to the j^{th} index is referred to as aᵢⱼ, and establish a judgment matrix A with n indexes.

$$A = \{ {( {a}_{ij} )}_{n \times n} \} = [ \begin{matrix}{a}_{11} & {a}_{12} & … & {a}_{1 n} \\ {a}_{21} & {r}_{22} & … & {a}_{2 n} \\ … & … & … & … \\ {a}_{n 1} & {a}_{n 2} & … & {a}_{nn}\end{matrix} ]$$

TABLE 5
1~9 Scales Method

| No. | Assignment | Hierarchy of Importance |
|---|---|---|
| 1 | 1 | It means i and j are equally important. |
| 2 | 3 | It means i is a little more important than j. |
| 3 | 5 | It means i is obviously more important than j. |
| 4 | 7 | It means i is strongly more important than j. |
| 5 | 9 | It means i is extremely more important than j. |
| 6 | 2, 4, 6, 8 | It means a medium among the 1~9 scales. |
| 7 | reciprocal | If the relative importance ratio between xᵢ and xⱼ |
| | | is aᵢⱼ, then the relative importance ratio between |
| | | xⱼ and xᵢ is aⱼᵢ = 1/aᵢⱼ. |

Second, normalize the judgment matrix, calculate the weight w of each evaluation index uᵢⱼ, and calculate the maximum characteristic root and the corresponding eigenvector of the judgment matrix.

Third, conduct consistency test of the judgment matrix. If the matrix passes the test, the eigenvector is the weight vector of indexes. If not, a new judgment matrix should be re-established. The weights of quality evaluation indexes for dehydrated instant rice are shown in Table 3.

S5: To determine the membership of evaluation indexes, establish fuzzy evaluation matrixes:

First, divide the evaluation indexes in the Table 3 into ascending quantitative evaluation indexes, appropriate interval quantitative indexes, and descending quantitative indexes, wherein luster, morphology, appearance before reconstitution, appearance, mouthfeel, and aroma after reconstitution, and texture characteristics, namely cohesiveness, chewiness, elasticity are all ascending quantitative evaluation indexes. Meanwhile, adhesiveness and hardness are descending quantitative indexes.

Second, calculate the membership of each second-level evaluation indexes, wherein for the ascending quantitative index, the general membership function for the corresponding level is:

$$r = \{ \begin{matrix}0 & {x}_{ij} \leq min ( {x}_{ij} ) \\ \frac{{x}_{ij} - min ( {x}_{ij} )}{max ( {x}_{ij} ) - min ( {x}_{ij} )} & min ( {x}_{ij} ) < {x}_{ij} < max ( {x}_{ij} ) \\ 1 & {x}_{ij} \geq max ( {x}_{ij} )\end{matrix}$$

For the descending quantitative index, the general membership function is:

$$r = \{ \begin{matrix}1 & {x}_{ij} \leq min ( {x}_{ij} ) \\ \frac{max ( {x}_{ij} ) - {x}_{ij}}{max ( {x}_{ij} ) - min ( {x}_{ij} )} & min ( {x}_{ij} ) < {x}_{ij} < max ( {x}_{ij} ) \\ 0 & {x}_{ij} \geq max ( {x}_{ij} )\end{matrix}$$

Based on the above, establish a single factor fuzzy matrix R:

$$R = \{ {r}_{ij} \} = [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ]$$

S6: To conduct fuzzy matrix composite operations by using the operator M(⋅, ⊕), obtain a fuzzy comprehensive evaluation value.

$$Y = w ( \cdot , ⊕ ) R = [ \begin{matrix}{w}_{1} & {w}_{2} & {w}_{3}\end{matrix} ] ( \cdot , ⊕ ) [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ]$$

The comprehensive evaluation values of eighty-eight samples of raw materials are shown in Table 6.

S7: The mathematical model between the comprehensive evaluation value of instant rice and the properties of raw material is obtained by applying stepwise regression analysis, namely:

Y=0.635×10⁻⁵·x₁^{0.365}·x₂^{0.207}=x₃^{0.334}·x₄^{0.952}·x₅^{1.225} (Eq. 1)

wherein, x₁ represents the moisture; x₂ represents the content of amylose; x₃ represents the content of protein; x₄ represents the taste value; x₅ represents the gelatinization temperature.

TABLE 6
Comprehensive Evaluation Values (CE Value) of Different
Rice Variety for Producing Dehydrated Instant Rice

| No | Sample Name | CE Value (Y) |
|---|---|---|
| 01 | 00884 | 0.581 |
| 02 | 8414 Nuo | 0.322 |
| 03 | K You 6 | 0.758 |
| 04 | Q Nuo 2 | 0.300 |
| 05 | R4132 Nuo | 0.236 |
| 06 | T You 100 | 0.626 |
| 07 | T You 1128 | 0.723 |
| 08 | T You 115 | 0.659 |
| 09 | T You 207 | 0.581 |
| 10 | T You 272 | 0.728 |
| 11 | T You 597 | 0.678 |
| 12 | Y Liangyou 1 | 0.670 |
| 13 | Y Liangyou 7 | 0.656 |
| 14 | Baofeng 2 | 0.744 |
| 15 | Bo II You15 | 0.869 |
| 16 | Boyou Shuangqing | 0.831 |
| 17 | Changguidao | 0.790 |
| 18 | Changnuo 468 | 0.298 |
| 19 | Chengnuo 88 | 0.257 |
| 20 | Enuo 9 | 0.256 |
| 21 | Fujing 2103 | 0.734 |
| 22 | Ganxin 203 | 0.438 |
| 23 | Guichao | 0.725 |
| 24 | Guiyou 2 | 0.577 |
| 25 | Hongnuo 19 | 0.264 |
| 26 | Jijing 94 | 0.769 |
| 27 | Jinongda 45 | 0.766 |
| 28 | Jinyou 163 | 0.751 |
| 29 | Jinyou 967 | 0.582 |
| 30 | Jinyou 978 | 0.536 |
| 31 | Jindao 8 | 0.753 |
| 32 | Jingxian 89 | 0.722 |
| 33 | Liangyou 6326 | 0.659 |
| 34 | Liaoxing 1 | 0.748 |
| 35 | Maoyou 601 | 0.601 |
| 36 | Meichi 8 | 0.573 |
| 37 | Mengliangyou 838 | 0.552 |
| 38 | Qianyou107 | 0.515 |
| 39 | Qianyou 568 | 0.602 |
| 40 | Qiuguang | 0.740 |
| 41 | Rongyou 7 | 0.318 |
| 42 | Rongyou 9 | 0.665 |
| 43 | Shenyou 9723 | 0.680 |
| 44 | Shenyou 9734 | 0.549 |
| 45 | Shenxian 6 | 0.750 |
| 46 | Shugeng | 0.775 |
| 47 | Tianfengyou 316 | 0.551 |
| 48 | Tianyou 103 | 0.561 |
| 49 | Tianyou 122 | 0.506 |
| 50 | Tianyou 998 | 0.600 |
| 51 | Wanjinyou 122 | 0.664 |
| 52 | Weiyou 277 | 0.613 |
| 53 | Weiyou 46 | 0.649 |
| 54 | Weiyou 644 | 0.623 |
| 55 | Weiyou 647 | 0.553 |
| 56 | Weiyouwan 3 | 0.637 |
| 57 | Wufengyou 998 | 0.571 |
| 58 | Wufengyou T025 | 0.654 |
| 59 | Wuyou 308 | 0.615 |
| 60 | Xianhe Dali | 0.732 |
| 61 | Xianong 2 | 0.770 |
| 62 | Xiangnuo | 0.266 |
| 63 | Xiangzaoxian 06 | 0.342 |
| 64 | Xiangzaoxian 24 | 0.534 |
| 65 | Xiangzaoxian 32 | 0.453 |
| 66 | Xiangzaoxian 42 | 0.534 |
| 67 | Xiangzaoxian 45 | 0.433 |
| 68 | Xindai | 0.733 |
| 69 | Xinnian 3 | 0.441 |
| 70 | Yangdao 6 | 0.749 |
| 71 | You 1686 | 0.511 |
| 72 | Youyou 128 | 0.742 |
| 73 | Yueyou 72 | 0.710 |
| 74 | Yunhui 290 | 0.750 |
| 75 | Zhanyou 2009 | 0.741 |
| 76 | Zhanyou 226 | 0.865 |
| 77 | Zhanyou 809 | 0.800 |
| 78 | Changbai 9 | 0.746 |
| 79 | Changbai 9 | 0.771 |
| 80 | Zhefu 802 | 0.414 |
| 81 | Zhennuo | 0.253 |
| 82 | Zhennuo 4130 | 0.265 |
| 83 | Zhong 9 You 838 | 0.296 |
| 84 | Zhongjiazao 32 | 0.363 |
| 85 | Zhongyou 106 | 0.614 |
| 86 | Zhongyou 9918 | 0.803 |
| 87 | Zhuliangyou 176 | 0.489 |
| 88 | Zhuliangyou 819 | 0.374 |

### Embodiment 2

The comprehensive evaluation values of the fresh wet rice noodles, rice sponge cakes and glue puddings produced by eighty-eight different rice samples are obtained through the same procedure as stated in the Embodiment 1, the statistics are shown in Table 7.

TABLE 7
Statistics of the Comprehensive Evaluation Values (CEV) of Different
Rice Varieties for Producing Different Rice Products

| | Mean | SD | Max | Min | Range |
|---|---|---|---|---|---|
| CEV of Fresh Wet Rice Noodle | 0.596 | 0.175 | 0.863 | 0.204 | 0.659 |
| CEV of Rice Sponge Cake | 0.601 | 0.188 | 0.853 | 0.143 | 0.710 |
| CEV of Glue Pudding | 0.513 | 0.191 | 0.897 | 0.125 | 0.772 |

The mathematical models between the comprehensive evaluation values of fresh wet rice noodles, rice sponge cakes, as well as glue puddings, and the characteristics of raw materials are obtained by conducting stepwise regression analysis.

Fresh Wet Rice Noodle:

Y=2.80×10⁻⁴·x₁^{0.322}·x₂^{1.571} (Eq. 2)

wherein, x₁ represents the content of amylose, and x₂ represents the gelatinization temperature.

Rice Sponge Cakes:

Y=1.06×10⁻⁴·x₁^{0.372}·x₂^{-0.365}·x₃^{1.833} (Eq. 3)

wherein, x₁ represents the content of amylose; x₂ represents the peak time; x₃ represents the gelatinization temperature.

Glue Puddings:

Y=329.55·x₁^{0.842}·x₂^{-0.158}·x₃^{-1.885} (Eq. 4)

wherein, x₁ represents the moisture; x₂ represents the content of amylose; x₃ represents the gelatinization temperature.

The verification of the mathematical model between the comprehensive evaluation values of rice products and the characteristics of raw materials includes:

S1: Measure the physicochemical indexes of fifteen types of raw rice materials; measure the moisture, and the content of protein and amylose with a grain near-infrared analyzer; measure the taste value with a taste meter; measure the gelatinization properties and obtain parameters of gelatinization properties such as gelatinization temperature (GTP), peak viscosity (PV), disintegration value (DV), minimum viscosity (MV), retrogradation (RG), and final viscosity (FV), as shown in the Table 8.

TABLE 8
Physicochemical Indexes of Fifteen Types of Raw Rice Materials

| | | | | | PV | MV | AV | FV | RG | PT | GTP |
|---|---|---|---|---|---|---|---|---|---|---|---|
| No. | M | A | P | TV | (Pa · s) | | | | | (min) | □ |
| 1 | 13.20 | 16.54 | 10.10 | 47.00 | 3.70 | 1.95 | 1.74 | 3.38 | 0.32 | 5.80 | 80.70 |
| 1 | 12.20 | 17.32 | 10.20 | 46.00 | 3.50 | 1.85 | 1.65 | 1.98 | 0.33 | 5.95 | 80.60 |
| 2 | 11.40 | 20.80 | 9.20 | 42.00 | 3.48 | 2.23 | 1.25 | 1.99 | 0.74 | 5.98 | 80.20 |
| 3 | 12.40 | 17.80 | 9.90 | 52.00 | 2.78 | 1.77 | 1.01 | 1.83 | 0.82 | 6.18 | 79.78 |
| 4 | 13.10 | 2.54 | 8.43 | 48.00 | 3.64 | 1.69 | 1.95 | 2.90 | 0.95 | 5.56 | 67.75 |
| 5 | 11.89 | 1.89 | 9.01 | 47.00 | 3.87 | 1.72 | 2.15 | 2.78 | 0.63 | 5.51 | 67.65 |
| 6 | 13.40 | 16.20 | 8.60 | 68.00 | 2.75 | 1.82 | 0.93 | 1.96 | 1.03 | 6.08 | 83.60 |
| 7 | 12.23 | 17.60 | 6.48 | 43.00 | 2.05 | 0.58 | 1.47 | 1.95 | 0.48 | 6.17 | 80.65 |
| 8 | 12.10 | 23.60 | 10.40 | 40.00 | 2.70 | 1.97 | 0.73 | 1.63 | 0.90 | 6.27 | 82.30 |
| 9 | 13.40 | 18.70 | 9.50 | 69.00 | 2.69 | 1.89 | 0.80 | 1.79 | 0.99 | 6.07 | 77.55 |
| 10 | 12.20 | 16.90 | 10.60 | 40.00 | 3.87 | 1.98 | 1.89 | 2.76 | 0.87 | 5.37 | 83.95 |
| 11 | 10.60 | 21.70 | 10.90 | 44.00 | 2.64 | 1.73 | 0.91 | 1.61 | 0.70 | 5.57 | 80.75 |
| 12 | 12.60 | 24.20 | 10.80 | 36.00 | 0.44 | 0.10 | 0.34 | 0.53 | 0.19 | 5.33 | 79.40 |
| 13 | 10.67 | 11.80 | 10.41 | 46.00 | 3.15 | 1.83 | 1.33 | 3.24 | 1.02 | 5.89 | 72.85 |
| 14 | 11.50 | 1.10 | 10.23 | 42.00 | 4.59 | 2.49 | 2.10 | 3.01 | 0.91 | 5.73 | 66.99 |
| 15 | 11.93 | 10.40 | 9.56 | 47.50 | 3.65 | 2.18 | 1.47 | 3.16 | 0.85 | 5.65 | 71.40 |

M = Moisture
A = Amylose
P = Protein
TV = Taste Value
AV = Attenuation Value
PT = Peak Time

S2: Substitute aforementioned physicochemical indexes of raw rice materials into the mathematical models shown as Eq. 1, Eq. 2, Eq. 3 and Eq. 4, respectively, and the estimated comprehensive evaluation values of the corresponding rice products are shown in the Table 9.

TABLE 9
Estimated Comprehensive Evaluation Values of Processed
Rice Products of Fifteen Types of Raw Rice Materials

| | Instant | Fresh & Wet | Rice Sponge | Glue |
|---|---|---|---|---|
| No. | Rice | Rice Noodle | Cake | Pudding |
| 01 | 0.609 | 0.684 | 0.617 | 0.473 |
| 1 | 0.586 | 0.693 | 0.621 | 0.440 |
| 2 | 0.523 | 0.730 | 0.657 | 0.408 |
| 3 | 0.651 | 0.688 | 0.607 | 0.453 |
| 4 | 0.318 | 0.284 | 0.225 | 0.878 |
| 5 | 0.288 | 0.258 | 0.201 | 0.850 |
| 6 | 0.858 | 0.718 | 0.644 | 0.449 |
| 7 | 0.475 | 0.697 | 0.617 | 0.439 |
| 8 | 0.563 | 0.791 | 0.711 | 0.400 |
| 9 | 0.843 | 0.669 | 0.590 | 0.506 |
| 10 | 0.544 | 0.733 | 0.690 | 0.409 |
| 11 | 0.573 | 0.748 | 0.694 | 0.376 |
| 12 | 0.503 | 0.754 | 0.712 | 0.441 |
| 13 | 0.457 | 0.523 | 0.447 | 0.505 |
| 14 | 0.236 | 0.213 | 0.159 | 0.917 |
| 15 | 0.453 | 0.486 | 0.417 | 0.588 |

S3: Process aforementioned raw rice materials into dehydrated instant rice; measure the texture indexes (hardness, adhesiveness, elasticity, chewiness, and cohesiveness) and the organoleptic qualities (luster before reconstitution, appearance, mouthfeel, and aroma after reconstitution) based on the indexes in the quality evaluating system; calculate the membership of different indexes; calculate the measured comprehensive evaluation values based on the membership and weight of each index, as shown in the Table 10.

S4: Process aforementioned raw rice materials into fresh wet rice noodles; measure the texture indexes (elasticity, viscosity, hardness, and chewiness) and organoleptic qualities (luster, aroma, morphology, and mouthfeel) and other physicochemical indexes (pulping value and broken rate) based on the indexes in the quality evaluating system; calculate the membership of each index; calculate the measured comprehensive evaluation values based on the membership and weight of each index, as shown in the Table 10.

S5: Process aforementioned raw rice materials into rice sponge cakes, measure the texture indexes (elasticity, viscosity, hardness, resilience, and chewiness) and organoleptic qualities (morphology, luster, aroma, taste and mouthfeel) and other physicochemical indexes (specific volume and titration acidity) based on the indexes in the quality evaluating system; calculate the membership of different indexes, calculate the measured comprehensive evaluation values based on the membership and weight of each index, as shown in the Table 10.

S6: Process aforementioned raw rice materials into glue pudding; measure the texture indexes (elasticity, viscosity, hardness, resilience, and chewiness) and organoleptic qualities (appearance, taste and turbidity) and other physicochemical indexes (frozen cracking rate, dehydration rate, and soup lucidity) based on the indexes in the quality evaluating system; calculate the measured comprehensive evaluation values based on the membership and weight of each index, as shown in the Table 10.

TABLE 10
Comprehensive Evaluation Values of Fifteen Types
of Rice Raw Materials for Producing Rice Products

| | Instant | Fresh & Wet | Rice Sponge | Glue |
|---|---|---|---|---|
| No. | Rice | Rice Noodle | Cake | Pudding |
| 01 | 0.581 | 0.677 | 0.632 | 0.442 |
| 1 | 0.614 | 0.695 | 0.612 | 0.438 |
| 2 | 0.497 | 0.739 | 0.573 | 0.396 |
| 3 | 0.663 | 0.702 | 0.565 | 0.475 |
| 4 | 0.298 | 0.243 | 0.204 | 0.865 |
| 5 | 0.305 | 0.244 | 0.185 | 0.865 |
| 6 | 0.853 | 0.703 | 0.612 | 0.487 |
| 7 | 0.534 | 0.725 | 0.545 | 0.544 |
| 8 | 0.541 | 0.755 | 0.705 | 0.364 |
| 9 | 0.865 | 0.643 | 0.534 | 0.534 |
| 10 | 0.487 | 0.673 | 0.573 | 0.465 |
| 11 | 0.554 | 0.746 | 0.704 | 0.265 |
| 12 | 0.489 | 0.724 | 0.736 | 0.353 |
| 13 | 0.449 | 0.498 | 0.398 | 0.626 |
| 14 | 0.259 | 0.247 | 0.189 | 0.870 |
| 15 | 0.488 | 0.468 | 0.397 | 0.636 |

S7: Compare the estimated comprehensive quality values of rice products in the S2 with the measured comprehensive quality values of rice products in the S3˜S6; analyze the correlation between the estimated values and measured values. If their correlation coefficient is no less than 0.9, it indicates that the prediction ability of the model is satisfactory.

To predict the processing suitability of the raw rice material Jinyou 402 into rice products includes:

S1: measure the physicochemical indexes of the raw rice material Jinyou 402. Being measured by the grain near-infrared analyzer, the moisture of the raw rice material is 12.56%, the content of protein and amylose are 9.42% and 24.63%, respectively. Being measured by the taste meter, the taste value is 39; the gelatinization temperature (GTP) is 84.43° C., the peak viscosity (PV) is 2.61 Pa·s, the attenuation value (AV) is 1.21 Pa·s, the minimum viscosity (MV) 1.39 Pa·s, the retrogradation (RG) 1.15 Pa·s, the final viscosity (FV) 2.52 Pa·s, and the peak time is 5.7 min.

S2: Substituted the aforementioned indexes of raw rice materials into the mathematical models Eq. 1, Eq. 2, Eq. 3 and Eq. 4, respectively, and the estimated comprehensive evaluation values of dehydrated instant rice, fresh wet rice noodles, rice sponge cakes, and glue puddings made from Jinyou 402 are 0.562, 0.835, 0.785 and 0.391, accordingly. Therefore, it is known that the Jinyou 402 is most suitable for producing fresh wet rice noodles, while it is not suitable to be produced to rice sponge cakes, glue puddings, and dehydrated instant rice.

1. A rapid screening method of processing raw materials for rice products comprising:
(1) collecting representative raw materials samples;
(2) measuring physicochemical indexes of different varieties of raw materials;
(3) producing rice products with different varieties of raw materials;
(4) according to characteristics of each type of rice products, establishing an index set of multi-level assessment of the quality of rice products and obtaining weights of different indexes by utilizing analytic hierarchy process (AHP);
(5) determining a membership of each evaluating index and constructing a fuzzy evaluation matrix;
(6) obtaining fuzzy comprehensive evaluation values by conducting fuzzy matrix composite operations;
(7) obtaining a mathematical model between the comprehensive evaluation values of rice products and the characteristics of raw materials by conducting regression analyses; and
(8) predicting processing suitability of different varieties of raw materials to produce rice products by adopting the mathematical model in the step (7).

2. A rapid screening method of processing raw materials for rice products according to claim 1, wherein in the step (2), the physicochemical indexes of different varieties of raw materials comprises:
\textcircled{1} moisture, contents of protein, and amylose of raw materials, measured by a grain near infrared analyzer;
\textcircled{2} taste values of raw materials measured by a taste meter;
\textcircled{3} gelatinization parameters through gelatinization tests after grinding and sifting.

3. A rapid screening method of processing raw materials for rice products according to claim 2, wherein the step (4), according to the characteristics of each type of rice products, comprises:
\textcircled{1} establishing an index set of multi-level assessment of the quality of rice products, including the rice products including rice noodles, instant rice, glue puddings, and rice sponge cakes;
\textcircled{2} classifying the indexes for evaluating rice products quality into multi-level evaluation indexes;
\textcircled{3} dividing first-level evaluation indexes into organoleptic quality index U₁, texture index U₂, and other physicochemical index U₃;
\textcircled{4} obtaining second-level assessment indexes Uᵢⱼ below the first-level evaluation indexes Uᵢ, according to the characteristics of the rice noodles:
the second-level evaluation indexes of the organoleptic quality index including: luster U₁₁, aroma U₁₂, morphology U₁₃, and mouthfeel U₁₄, namely, U₁={U₁₁, U₁₂, U₁₃, U₁₄},
the second-level evaluation indexes of the texture index including: elasticity U₂₁, viscidity U₂₂, hardness U₂₃, and chewiness U₂₄, namely, U₂={U₂₁, U₂₂, U₂₃, U₂₄};
the second-level evaluation indexes of the other physicochemical indexes including: pulping value U₃₁, broken rate U₃₂, namely, U₃={U₃₁, U₃₂};
\textcircled{5} obtaining the second-level evaluation indexes Uᵢⱼ under the first-level evaluation indexes Uᵢ, according to the characteristics of the instant rice:
the second-level evaluation indexes of the organoleptic quality index including: luster before reconstitution U₁₁, morphology before reconstitution U₁₂, appearance U₁₃, mouthfeel U₁₄, and aroma U₁₅ after reconstitution, namely, U₁={U₁₁, U₁₂, U₁₃, U₁₄, U₁₅};
the second-level evaluation indexes of the texture index including: hardness U₂₁, adhesiveness U₂₂, elasticity U₂₃, chewiness U₂₄, and cohesiveness U₂₅, namely, U₂={U₂₁, U₂₂, U₂₃, U₂₄, U₂₅},
\textcircled{6} obtaining the second-level evaluation indexes Uᵢⱼ under the first-level evaluation indexes Uᵢ, according to the characteristics of the glue puddings:
the second-level evaluation indexes of the organoleptic quality index including: appearance U₁₁, mouthfeel U₁₂, and soup cloudiness U₁₃, namely, U₁={U₁₁, U₁₂, U₁₃};
the second-level evaluation indexes of the texture index including: hardness U₂₁, elasticity U₂₂, adhesiveness U₂₃, resilience U₂₄, and chewiness U₂₅, namely, U₂={U₂₁, U₂₂, U₂₃, U₂₄, U₂₅},
the second-level assessment indexes of the other physicochemical indexes including: frozen cracking rate U₃₁, dehydration rate U₃₂, soup lucidity rate U₃₃, namely, U₃={U₃₁, U₃₂, U₃₃};
\textcircled{7} obtaining the second-level assessment indexes Uᵢⱼ under the first-level assessment indexes Uᵢ, according to the characteristics of the rice sponge cakes:
the second-level assessment indexes of the organoleptic quality index including: morphology U₁₁, luster U₁₂, and aroma U₁₃, taste U₁₄, and mouthfeel U₁₅, namely, U₁={U₁₁, U₁₂, U₁₃, U₁₄, U₁₅};
the second-level assessment indexes of the texture index including: hardness U₂₁, elasticity U₂₂, adhesiveness U₂₃, resilience U₂₄, and chewiness U₂₅, namely, U₂={U₂₁, U₂₂, U₂₃, U₂₄, U₂₅};
the second-level assessment indexes of the other physicochemical indexes including: specific volume U₃₁ and titration acidity U₃₂, namely, U₃={U₃₁, U₃₂}.

4. A rapid screening method of processing raw materials for rice products according to claim 3, wherein in the step (4), obtaining weights of different evaluation indexes by conducting analytical hierarchy process (AHP) further comprises:
according to experts' scoring results, obtaining judgment matrixes of the second-level evaluation index and the first-level evaluation index,
normalizing the judgment matrixes,
calculating second-weight of the second-level evaluation indexes wᵢⱼ and first-weight of the first evaluation indexes wᵢ, and
obtaining weight sets w₁={w₁₁, w₁₂, w₁₃, . . . , w₁ⱼ}, w₂={w₂₁, w₂₂, w₂₃, . . . , w₂ⱼ}, w₃={w₃₁, w₃₂, w₃₃, . . . , w₃}, w={w₁, w₂, w₃, . . . , wᵢ}.

5. A rapid screening method of processing raw materials for rice products according to claim 4, wherein in the step (5), determining memberships of each evaluation index, establishing fuzzy evaluation matrixes further comprises:
\textcircled{1} dividing the evaluation indexes in the step (4) further into ascending quantitative evaluation indexes, appropriate interval quantitative indexes, and descending quantitative indexes;
\textcircled{2} calculating memberships of each second-level indexes; wherein:
for the ascending quantitative indexes, the general membership function for the corresponding level is:
$$r = \{ \begin{matrix}0 & {x}_{ij} \leq min ( {x}_{ij} ) \\ \frac{{x}_{ij} - min ( {x}_{ij} )}{max ( {x}_{ij} ) - min ( {x}_{ij} )} & min ( {x}_{ij} ) < {x}_{ij} < max ( {x}_{ij} ) \\ 1 & {x}_{ij} \geq max ( {x}_{ij} )\end{matrix} ;$$
for the descending quantitative index, the general membership function for the corresponding level is:
$$r = \{ \begin{matrix}1 & {x}_{ij} \leq min ( {x}_{ij} ) \\ \frac{max ( {x}_{ij} ) - {x}_{ij}}{max ( {x}_{ij} ) - min ( {x}_{ij} )} & min ( {x}_{ij} ) < {x}_{ij} < max ( {x}_{ij} ) \\ 0 & {x}_{ij} \geq max ( {x}_{ij} )\end{matrix}$$
for the appropriate interval quantitative index, the general membership function for the corresponding level is:
$$r = \{ \begin{matrix}1 & {s}_{1} \leq {x}_{ij} \leq {s}_{2} \\ \frac{{x}_{ij} - min ( {x}_{ij} )}{{s}_{1} - min ( {x}_{ij} )} & min ( {x}_{ij} ) < {x}_{ij} < {s}_{1} \\ \frac{max ( {x}_{ij} ) - {x}_{ij}}{max ( {x}_{ij} ) - {s}_{2}} & {s}_{2} < {x}_{ij} < max ( {x}_{ij} ) \\ 0 & {x}_{ij} > max ( {x}_{ij} ) , {x}_{ij} < min ( {x}_{ij} ) ,\end{matrix}$$
wherein xᵢⱼ represents the measurements of the second-level indexes, min(xᵢⱼ) and max(xᵢⱼ) represent the minimum and maximum values, respectively, and s₁ and s₂ represent the lower limit of the best value and the upper limit of the best value, respectively; and
\textcircled{3} establishing a single factor fuzzy matrix R:
$$R = \{ {r}_{ij} \} = [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ] .$$

6. A rapid screening method of processing raw materials for rice products according to claim 5, wherein in the step (6), obtaining fuzzy comprehensive evaluation value by conducting fuzzy matrix composite operation further comprises a multiplication of the single factor fuzzy matrix and weights of indexes determined in the step (4),
$$Y = w ∘ R = [ \begin{matrix}{w}_{1} & {w}_{2} & {w}_{3}\end{matrix} ] ∘ [ \begin{matrix}{r}_{11} & {r}_{12} & … & {r}_{1 m} \\ {r}_{21} & {r}_{22} & … & {r}_{2 m} \\ … & … & … & … \\ {r}_{i 1} & {r}_{i 2} & … & {r}_{im}\end{matrix} ]$$
wherein ∘ represents operations, different fuzzy operators are adopted according to the situations and operation results.

7. A rapid screening method of processing raw materials for rice products according to claim 6, wherein in the step (6), using operator M(⋅, ⊕) in all calculations of the fuzzy matrix composite operations, ⋅ and ⊕ represent algebraic product and sum of the fuzzy set, respectively.

8. A rapid screening method of processing raw materials for rice products according to claim 7, wherein in step (7) further comprises obtaining the mathematical model Y=A·xᵢ^{b}^{ᵢ} between the comprehensive evaluation values of the rice products such as rice noodles, glue puddings, instant rice, and rice sponge cakes and the characteristics of raw materials, wherein Y represents comprehensive values of the rice products, A is type-related coefficient of the rice products, xᵢ represents the physicochemical indexes of raw materials, such as moisture, amylose, gelatinization temperature, and taste value; bᵢ represents exponents, i=1, 2, 3 . . . .

9. A rapid screening method of processing raw materials for rice products according to claim 8, wherein in the step (8), predicting the processing suitability of raw materials by using the mathematical model in the step (7) further comprises:
\textcircled{1} measuring the physicochemical indexes of raw materials, including using the grain near infrared analyzer to measure contents of moisture, protein and amylose of raw materials; obtaining the taste values with the taste meter; obtaining the gelatinization parameters, such as gelatinization temperatures, peak viscosity, disintegration values, minimum adhesiveness, retrogradation values, and final adhesiveness; and
\textcircled{2} substituting the aforesaid physicochemical indexes of raw materials into the mathematical model Y=A·xᵢ^{b}^{ᵢ} as defined in step (7), obtaining the comprehensive evaluation values of corresponding rice products quality, and evaluating the processing suitability of raw materials.
